(19) United States
(12) Patent Application Publication
(10) Pub. No.: US 2007/0134209 A1
(43) Pub. Date: Jun. 14, 2007

(54) CELLULAR ENCAPSULATION FOR SELF-ASSEMBLY OF ENGINEERED TISSUE

(75) Inventor: John S. Oakey, Boulder, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Metafluidics, Inc., Golden, CO (US)

(21) Appl. No.: 11/560,254

(22) Filed: Nov. 15, 2006

Related U.S. Application Data
(60) Provisional application No. 60/749,750, filed on Dec. 12, 2005.

(51) Int. Cl. A61K 35/12; A61K 9/50

(52) U.S. Cl. 424/93.7; 424/490

(57) ABSTRACT

Methods are disclosed of producing a cellular matrix for tissue self-assembly. Encapsulated living cells are provided, with each of the living cells separately encapsulated within a primary encapsulant. The encapsulated living cells are themselves encapsulated within a liquid or gel secondary encapsulant. The second encapsulant is polymerized.

### CROSS-REFERENCES TO RELATED APPLICATIONS

This application is a nonprovisional of, and claims the benefit of the filing date of, U.S. Prov. Pat. Appl. No. 60/749,750, entitled “CELLULAR ENCAPSULATION FOR SELF-ASSEMBLY OF ENGINEERED TISSUE,” filed Dec. 12, 2005 by John S. Oakey, the entire disclosure of which is incorporated herein by reference for all purposes

### BACKGROUND OF THE INVENTION

This application relates generally to self-assembly of structures. More specifically, this application relates to the use of self-assembly in tissue engineering.

There are numerous clinical presentations in which a patient has suffered damage to or loss of tissue. In many instances, the currently preferred treatment for such presentations is the use of autografts, sometimes in combination with any of a variety of mechanical devices used to maintain the position of the autograft tissue or underlying tissue during healing. The example of bone tissue provides an effective illustration. Complex load-bearing bone fractures are conventionally treated with a combination of autograft materials and fracture-fixation devices, which may be provided as screws, plates, associated hardware, and the like. In some instances, the damage to the bone tissue is sufficiently minor that the use of fixation devices may be avoided. This is true, for instance, in such cases as treating defects or void spaces that result from the removal of bone tumor or in treating bone loss in the alveolar ridge that results from periodontal disease. The use of bone grafts, and indeed of tissue grafts generally, suffers from a number of disadvantages. For example, autologous bone grafts are limited by graft availability and donor site morbidity.

An alternative approach to grafting makes use of “tissue engineering,” which refers more broadly to any method or process for creating biomaterials that contain living cells. Such materials find a diverse array of applications in different contexts—merely by way of illustration, examples include whole-cell biosensor arrays, vectors for targeted drug delivery, regenerative medicine, and the like. Conventional approaches to engineering tissue structures use may be characterized as “top down” bulk approaches. In these processes, cells are dispersed within a bulk homogeneous solution, which is processed to provide certain desired mechanical properties. Often, the bulk material begins as a liquid and is exposed to ultraviolet light, inducing polymerization or a phase change of the material to a solid. Alternatively, a solution such as liquid agar media may be heated to flow, and then later cooled for reversion back to a solid form. The kinetics of material erosion is designed to match the development of cells' natural extracellular support matrix. Development of this matrix depends on intercellular signaling, and therefore on the spatial distribution of cells and cell types. In the case of cartilage tissue, the matrix is typically composed of type-I collagen. The development of other tissues, particularly of functional tissues such as organ or muscle tissue, may be determined in large part by such factors as cellular spacing, interactions, etc. In the case of pluripotent stem cells, for instance, development into drastically different tissue types such as nerve versus endothelial tissue, may be dictated by local concentration.

There are a number of difficulties with such existing tissue-precursor formation techniques. First, the matrix material is homogeneous and monolithic, which limits the range of physical properties of the material; monolithic structures cannot readily be assembled in vivo and require invasive surgery to implant. Second, a bulk processing paradigm severely limits abilities to control the spatial distribution of cells within the matrix—this is a relevant consideration in the development of individual cells into functional tissue as well as in the differentiation of stem cells into functional tissue. Third, bulk processing severely limits any ability to colocalize cells with nutrients, growth factors, etc. within the matrix—this consideration is relevant because temporal control over the delivery of such products may be consequential to tissue development. Fourth, bulk techniques have poor compatibility with creation of material that may be injected arthroscopically.

There is accordingly a general need in the art for improved methods and systems of engineering tissues.

### BRIEF SUMMARY OF THE INVENTION

A first set of embodiments of the invention provide a method of producing a cellular matrix for tissue self-assembly. A plurality of encapsulated living cells are provided, with each of the living cells separately encapsulated within a primary encapsulant. The plurality of encapsulated living cells are themselves encapsulated within a secondary encapsulant. The secondary encapsulant is different from the primary encapsulant and comprises a liquid or gel. The second encapsulant is polymerized.

Examples of living cells that may be provided in the cellular matrix include mesenchymal stem cells, chondrocytes, osteoblasts, pancreatic islet cells, neuroprogenitor cells, and mynfibroblasts. In some instances, the plurality of encapsulated living cells are provided by separately microfluidically encapsulating the living cells within the primary encapsulant. For instance, each of the living cells might be encapsulated within a liquid droplet, with a phase change being induced to produce a solid particle. The encapsulated living cells may also be substantially monodisperse. For example, in one embodiment the plurality of encapsulated living cells comprise a set of volumes having at least 90% of a size distribution lying within 5% of a median size of the set of volumes. In specific embodiments, the plurality of encapsulated living cells comprise a set of substantially spherical volumes, each of the substantially spherical volumes having a diameter between about 10 μm and about 200 μm.

The polymerized second encapsulant may comprise an elastic material or may comprise a solid material in different embodiments. In some cases, the primary encapsulant comprises a hydrogel. One specific example for the primary encapsulant includes fibrin glue. In some instances, the secondary encapsulant may comprise a hydrogel. One specific example for the secondary encapsulant includes poly(ethylene glycol).

There are different ways of polymerizing the secondary encapsulant in different embodiments. For instance, in one embodiment, polymerizing the secondary encapsulant comprises photopolymerizing the secondary encapsulant. In other embodiments, the secondary encapsulant is polymerized thermally and/or chemically.

The plurality of encapsulated living cells may also form different structures within the secondary encapsulant in different embodiments. For instance, the may form an ordered periodic structure such as a two-dimensional periodic structure or a three-dimensional periodic structure, or they may form a nonperiodic structure. In some instances, the plurality of encapsulated living cells comprise a first plurality of a first kind of encapsulated living cells and a second plurality of a second kind of encapsulated living cells.

A second set of embodiments of the invention provide a cellular matrix. The cellular matrix comprises a plurality of encapsulated living cells, each of which is separately encapsulated within a primary encapsulant. The cellular matrix also comprises a polymerized secondary encapsulant within which the plurality of encapsulated living cells are disposed, with the secondary encapsulant being different from the primary encapsulant.

Again, examples of the living cells that may be comprised by the cellular matrix include mesenchymal stem cells, chondrocytes, osteoblasts, pancreatic islet cells, neuroprogenitor cells, and mynfibroblasts. The plurality of encapsulated living cells may be substantially monodisperse, such as by comprising a set of volumes having at least 90% of a size distribution lying within 5% of a median size of the set of volumes. They may also comprise a set of substantially spherical volumes, each having a diameter between about 10 μm and 200 μm.

There are also a number of examples of different materials that may be comprised by the cellular matrix. For instance, the polymerized secondary encapsulant might comprise an elastic material or might comprise a solid material. Examples of materials for the primary encapsulant include a hydrogel and fibrin glue. Examples of materials for the secondary encapsulant include a hydrogel and poly(ethylene glycol). The plurality of encapsulated living cells may also form different structures, such as where they form an ordered periodic structure or form a nonperiodic structure.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components.

FIG. 1 is a flow diagram that summarizes methods of producing a cellular matrix for tissue self-assembly in various embodiments of the invention;

FIG. 2 provides a schematic illustration of how a cellular matrix is produced using the methods of FIG. 1;

FIG. 3 is a micrograph of a microfluidics device illustrating a hydrogel microsphere fabrication process in a particular embodiment of the invention;

FIGS. 4A and 4B provide illustrations of different intermediate array structures that may be fabricated when producing the cellular matrix using the methods of FIG. 1;

FIG. 5 provides a schematic illustration of different architectural size scales in tissues of biological bodies;

FIGS. 6A and 6B show chemical structures of degradable poly(ethylene glycol) hydrogel precursors used in certain embodiments of the invention; and

FIG. 7 is a graphical representation of a degradation profile of hydrogels used in producing the cellular matrix in certain embodiments.

### DETAILED DESCRIPTION OF THE INVENTION

1. Overview

Embodiments of the invention reject the “top down” approach of the prior art and instead make use of a “bottom up” approach to tissue engineering. This approach is partly enabled by using microfluidic processing to form cell-containing particles, which may be used to create monodisperse suspensions of polymer particles. Microfluidics is a versatile and powerful research tool. The small size of microfluidics devices and systems provides unique transport and interfacial properties and significant parallelization and high-throughput capacities that are exploited in embodiments of the invention. In particular, microfluidic flows are especially useful because they provide ultralaminar flows that allow for highly precise spatial control over fluids and the forces they exert.

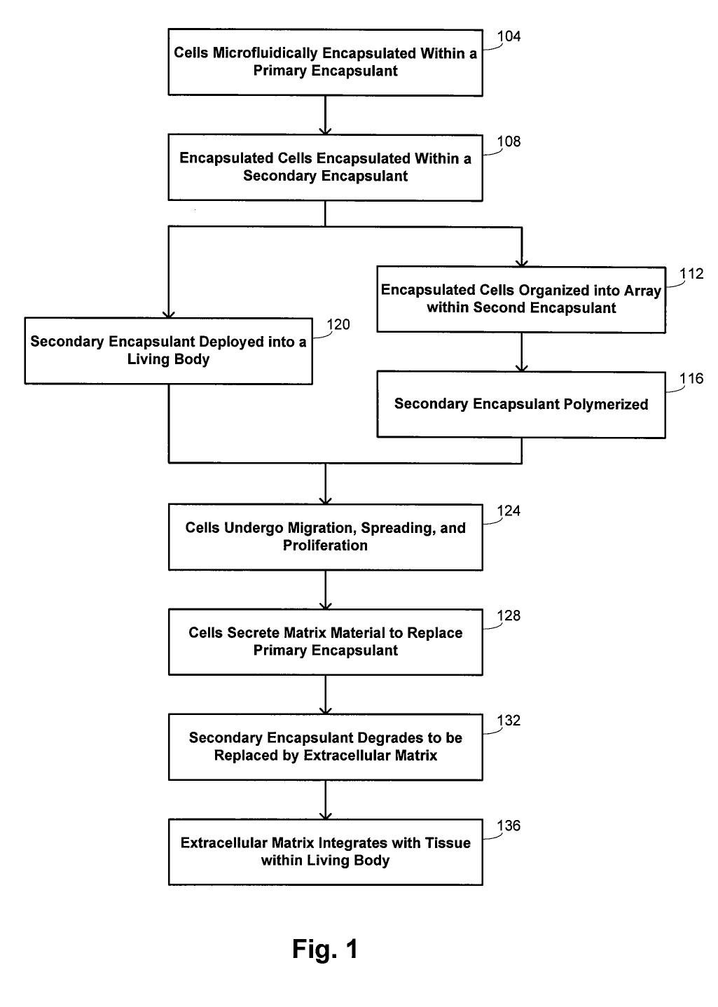

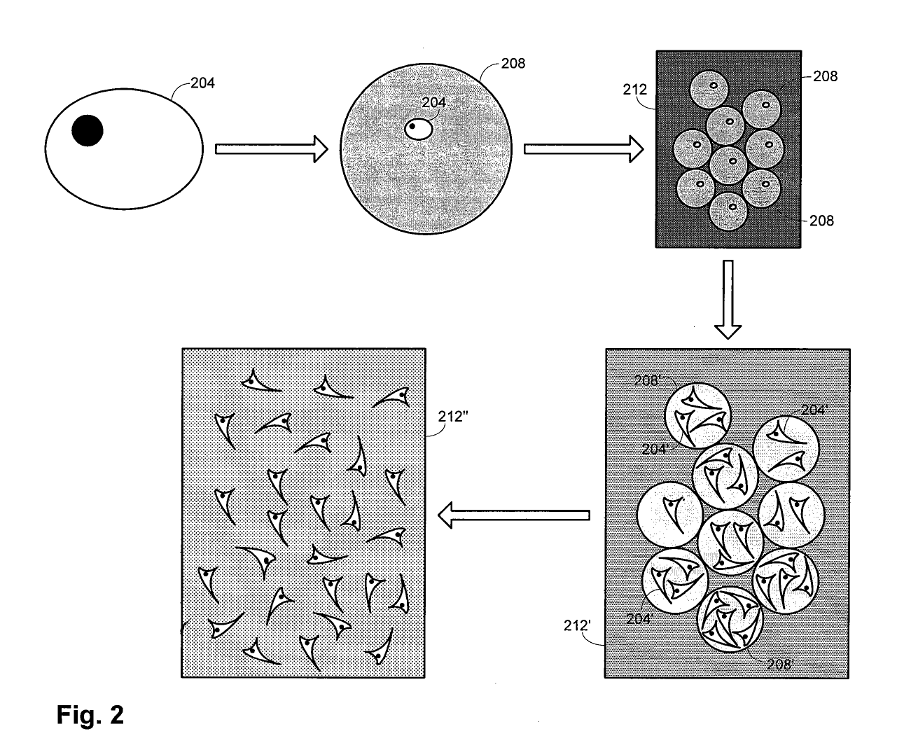

A general overview of methods of the invention is provided with the flow diagram of FIG. 1, which is discussed below in combination with FIG. 2, which provides a schematic illustration of how the methods of FIG. 1 may be implemented in a particular embodiment. To improve the clarity of illustration, the scale in FIG. 2 varies so that the relevant features may be readily identified in following the discussion. While the flow and schematic diagrams sometimes illustrate certain steps being performed in a particular order, this is not intended to be limiting. More generally, the steps indicated by the drawings may sometimes be performed in a different order. Furthermore, there may also be variation in the specific steps identified by the drawing: in some instances, not all of the indicated steps may be performed and in other instances, added steps not specifically identified in the drawings may additionally be performed.

The methods may begin at block 104 of FIG. 1 with cells 204 being microscopically encapsulated within a primary encapsulant 208. The primary encapsulant is degradable. The encapsulation is generally performed with a microfluidic device, one example of which is provided below. There are a number of specific techniques that may be used to achieve the encapsulation, one of which uses the microfluidic device to encapsulate single particles within a liquid droplet that is subsequently subjected to a change in physical conditions to induce a phase transition to form an encapsulating solid. The encapsulation volume may take different shapes, with certain embodiments providing substantially spherical encapsulation volumes having a diameter between about 10 μm and about 200 μm.

While many embodiments of the invention provide only a single cell within each encapsulation volume and use only cells of a single kind, neither of these is a requirement of the invention. In certain alternative embodiments, more than a single cell is provided within some or all of the encapsulation volumes; in such instances, it is not necessary that every encapsulation volume have the same number of cells. In other alternative embodiments, multiple kinds of cells may be used. Examples of different kinds of cells that may find utility in various tissue-engineering applications include mesenchymal stem cells, chondrocytes, osteoblasts, pancreatic islet cells, neuroprogenitor cells, and mynfibroblasts, among others.

The encapsulated cells are subjected to a second encapsulation at block 108. This produces a structure in which the encapsulating volumes 208 are themselves encapsulated within a secondary encapsulant 212, which is also degradable. The first and second encapsulants generally comprise different materials and may be selected for biological compatibility and to enable the formation of mesoscale architectures as described below. The secondary encapsulant is deployed into a living body at block 120. While this is shown in the drawing as occurring in parallel with certain other steps, it is often preferable to deploy the secondary encapsulant before some of those other steps are performed. Because a mixture of encapsulated cells within an unpolymerized secondary encapsulant may stil be fluid, it may be more easily deployed arthroscopically at block 120.

In a variety of different embodiments, the encapsulated cells may be organized into different array structures within the second encapsulant as indicated at block 112. This includes two- or three-dimensional ordered periodic structures and nonperiodic structures. The primary encapsulating volumes 208 may be formed as monodisperse particles in some embodiments. One criterion for monodispersity that is used in some embodiments is that at least 90% of a particle-size distribution of the particles lies within 5% of the median particle size. Such monodispersity is advantageous when the encapsulated cells are used in forming colloidal suspensions because the thermodynamic behavior of colloidal suspensions is predictable and relatively easily controlled with substantially uniform particle distributions. Monodisperse and binary colloidal systems display well-characterized fluid-solid coexistence behavior that may be tuned through such conditions as solvent ionic strength, surface charge, and composition. Colloidal crystallization via phase separation, sedimentation, dialysis, and other techniques is effective at creating highly ordered colloidal assemblies, which are formed in some embodiments by encapsulation within the second encapsulant at block 108. Specifically, such embodiments make use of colloidal self-assembly techniques to produce the structure as a colloidal gel or fractal aggregate. Colloidal gels are characterized by the aggregation of particles into large disordered sample-spanning networks. The structure of such semirigid continuous three-dimensional networks is determined by the aggregation conditions, with the aggregate structure typically being characterized by a fractal dimension d_{f}. Colloidal networks having different fractal dimensions and volume fractions exhibit different rheological properties.

The structure may be polymerized at block 116 and deployed into a living body at block 116. There are a number of different techniques that may be used in different embodiments to effect the polymerization, including photopolymerization techniques, chemical polymerization techniques, and thermal polymerization techniques among others. The resulting polymerized material may have different physical properties in different embodiments, with it sometimes forming a rigid material and other times forming an elastomeric material. Polymerizing secondary encapsulant effectively locks in the structure of the cellular spheres and provides complete temporal and spatial control over this process. Further, the polymerized secondary encapsulant provides a monolithic support structure for cells as the primary encapsulant erodes. This polymerization may be accomplished without deleterious effects to the cells even if the monomers or solvent providing the secondary encapsulant are not cytocompatible since the cells remain protected within the primary encapsulant volumes.

The character of the structure permits the cells to undergo migration, spreading, and proliferation in situ as indicated at block 124. They may also undergo differentiation to form cells that will ultimately become integrated with tissue within the body. Because the primary encapsulant 208 is degradable, it changes structure over time, progressively being replaced by matrix material secreted by the proliferating cells, as indicated at block 128. The secondary encapsulant also degrades as indicated at block 132, although usually over a longer time period than the primary encapsulant. FIG. 2 shows one intermediate structure in which the cells 204′ have undergone proliferation and differentiation, and both the primary encapsulant 208′ and the secondary encapsulant 212′ have undergone some degradation. This process causes the primary encapsulating volumes within the secondary encapsulant to give way to a continuous, highly porous, interconnected cellular phase.

As this process continues, an extracellular matrix 212″ containing the differentiated cells 204′ is produced, permitting the extracellular matrix to integrate with tissue within the living body. This is indicated at block 136 of FIG. 1.

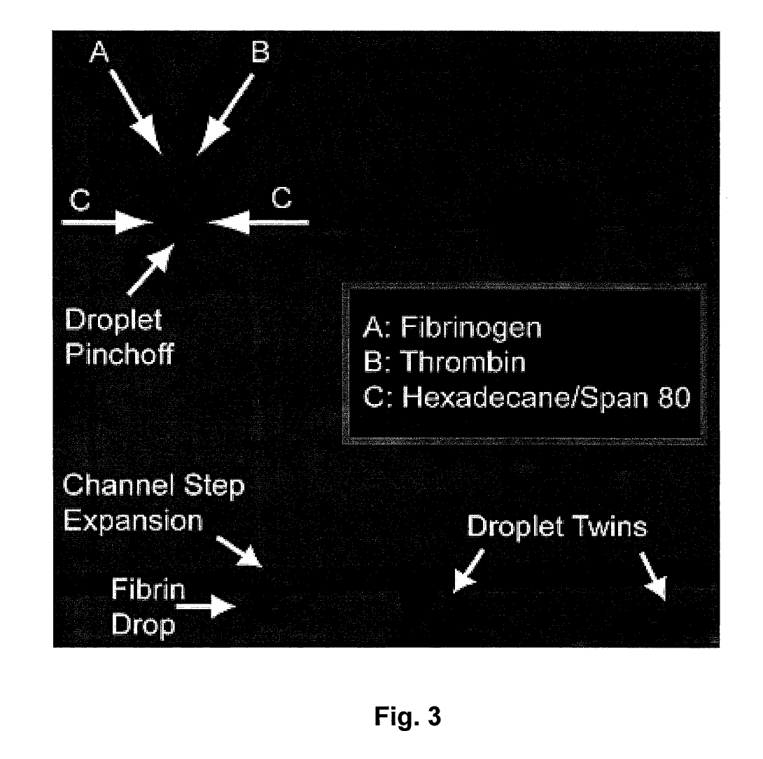

An example of a microfluidics device that may be used in encapsulation of the cells 204 with the primary encapsulant 208 is shown with the micrograph of FIG. 3. Precursors of the structure are shown flowing through channels A, B, and C to form droplets having the desired size. In the example shown, the primary encapsulant 208 comprises fibrin, which is one of the various encapsulant materials discussed below. The fibrin droplets in this example are formed with fibrinogen, thrombin, and hexadecane flows to the droplet pinch-off point in the manner described more fully below. Such microfluidics techniques are highly effective in producing monodisperse sets of droplets, resulting in the production of suspensions that are highly staple. The droplet size may be controlled during formation by manipulating the system's capillary number, which is defined as a ratio of viscous to interfacial stresses.

The example of FIG. 3 makes use of a cross-flow geometry, although other geometries may be used in alternative embodiments. For instance, suitable alternative geometries include hydrodynamic or flow focusing geometries, among others. In the cross-flow geometry illustrated in FIG. 3, the elongation and reconfiguration of the of the aqueous phase can be seen into either a channel-confined discoid slug or a free substantially spherical droplet. Monodisperse fibrin droplets have been created by the inventor using such a structure by dispensing aliquots of fibrinogen in a stoichiometric ratio. The miscible streams of fibrinogen and thrombin are stoichiometrically combined at a channel junction and immediately pinched by the hydrodynamic forces of the immiscible organic phase. An elongation of the interface results to balance the shear stress and discrete droplets are produced by the interfacial instability. In the initial channel, the droplets are confined by the walls to slug-like morphologies until, at the channel expansion, the droplet is allowed to fully relax and assume substantially spherical dimensions.

The collection of droplets thus formed into a suspension may be performed by extracting the droplets from the microfluidic device through a low-dead-volume-coupled syringe needle and sedimenting the droplets into a vial of hexadecane. The presence of an immiscible, more dense aqueous phase at the bottom of the vial serves to automatically separate the droplets from the organic fluid. This microscale process thus avoids the primary disadvantage of forming fibrin constructs on the macroscale, namely rapid gelation times. Since the droplet forms quickly, coalescence is resisted on the microscale. A typical time period for coagulation of the droplets into fibrin is less than about 30 seconds.

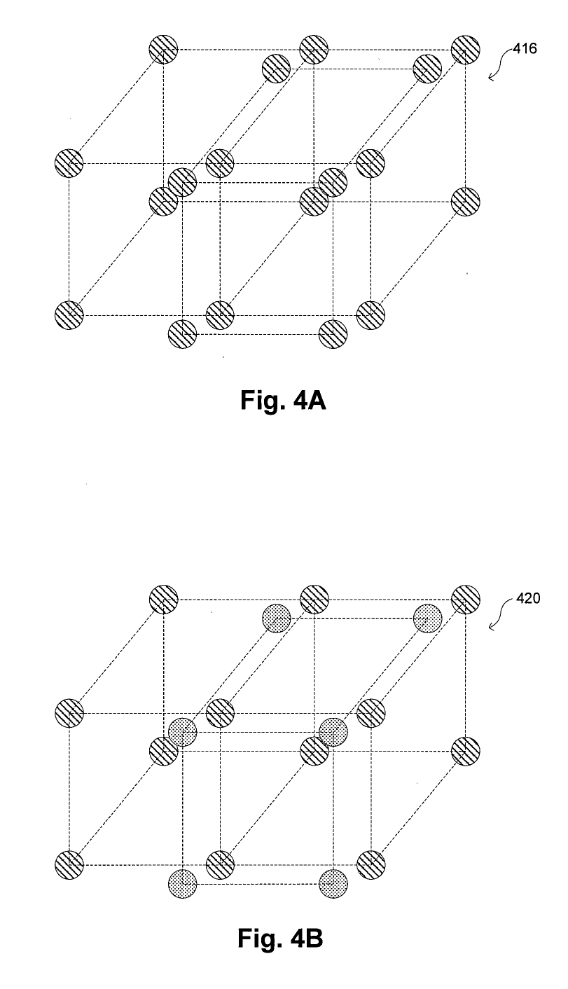

As previously noted, there are a variety of ordered, periodic, semiperiodic, nonperiodic or other structures that may be formed within the second encapsulant. One specific example of an ordered periodic structure that may be formed is illustrated in FIG. 4A as a body-centered-cubic (“bcc”) structure 416. Examples of semiperiodic and nonperiodic structures include glassy structures, quasicrystal structures, frozen-gel structures, and the like. Furthermore, while the structure 416 shown in FIG. 4A is illustrated as a unary structure having only a single type of particle, embodiments of the invention may more generally encompass structures having a plurality of types of particles. One such example is illustrated in FIG. 4B, also in the form of an ordered periodic bcc structure. For instance, one type of particle may comprise the cell-containing particles while another type of particle may comprise growth-factor-doped feeder particles created through either microchannel double-emulsion techniques or cocrystallization of binary, ternary, quaternary, etc. colloidal suspensions. More generally, the assembled structure may comprise any number of different types of particles in different embodiments, such as in embodiments where a further particle type is included to provide a functional response to or detect a change or event in the first type of particle; this may be particularly advantageous when the first type of particle includes a cell so that tissue is being engineered. While the illustrations of multiple particle types in FIG. 4B are provided for monodisperse and ordered periodic structures of substantially spherical particles, multiple particle types may also be included where the assembly in nonordered and/or where the particles are nonspherical.

2. Mesoscale Architecture

Embodiments of the invention incorporate mesoscale architecture in constructing the cellular matrix by using a multilevel embedded-encapsulation structure in which the internal encapsulants have a particular size. As previously noted, a suitable size for volumes of the primary encapsulant in providing a mesoscale architecture is between about 10 μm and about 200 μm.

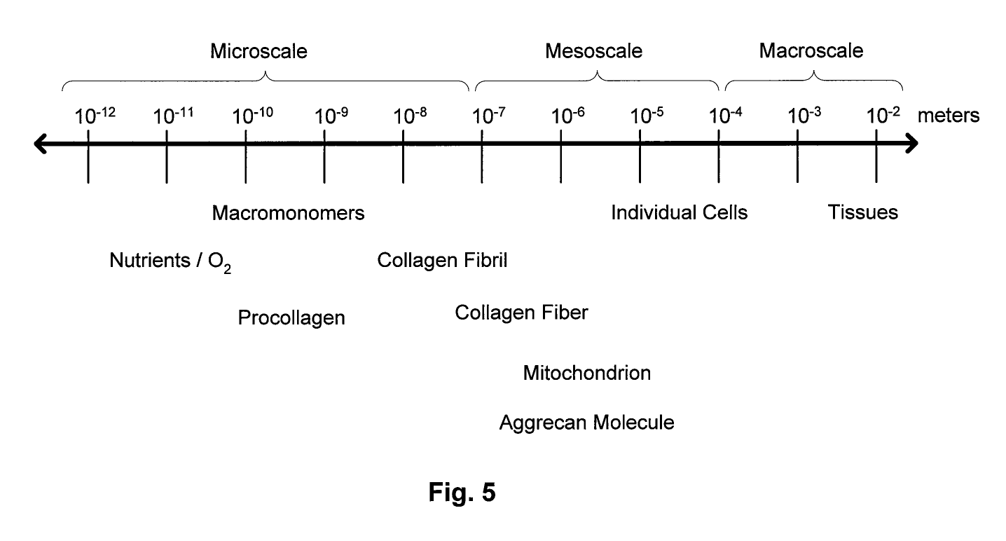

In considering the relevance of mesoscale architecture, it is worthwhile noting that structural size scales within the human body span a wide range, including extremely small nutrients such as glucose, oxygen, etc. at a scale of about 1-25 Å; proteins, polysaccharides, and nucleic acids at a scale of about 20-200 Å; macromolecular assemblies of individual proteins and protein subunits, such as collagen fibrils at a scale of 10-300 nm in diameter and microtubules at a scale of about 250 nm in diameter; intracellular components such as lysosomes and peroxisomes at a scale of 200-500 nm and mitochondria at a scale of about 3 μm; extracellular matrix components such as glycosaminoglycans and proteoglycans at a scale of about 1-5 μm and collagen fibers at a scale of about 0.5-3 μm; individual cells having a scale of about 5-160 μm; and aggregates of cells in the form of tissues. This structural diversity is summarized in FIG. 5, which also illustrates that use of the term “microscale” herein refers generally to the scale of structures formed by molecular interactions over tens and hundreds of nanometers; “macroscale” refers herein generally to the scale of structures visible to the naked eye such as aggregates of cells or tissues; and “mesoscale” refers herein to the scale intermediate between the microscale and the macroscale. The mesoscale thus encompasses what is sometimes referred to in the art as the “colloidal domain.”

Mesoscale architecture is relevant to a variety of different bodily tissue structures, functions, and development. For example, cortical bone contains at least six hierarchies of architectural control, including osteons, Haversian canals, collagen fibers, collagen fibrils, and collagen molecules, which are themselves triple helices of collagen α chains. Similar hierarchical control is seen in tendons. The inventor has discovered that providing control over mesoscale architecture facilitates the level of control over tissue development, cell interactions, and mechanical strength, while at the same time improving cell delivery and implantation.

There are at least four reasons why the incorporation of mesoscale structure may be relevant to tissue engineering scaffolds. First, effective diffusional transport is useful for cell-seeded biomaterials. In native tissues, diffusional limitations have been circumvented by the evolution of extensive mesoscale perfusion mechanisms, i.e. via blood vessels, capillaries, and venules. In this way, cells of the body may adequately exchange nutrients, gases, and wastes across optimized diffusional length scales. Biomaterial suitable for the culture of cells also advantageously provides effective diffusional properties. Materials that limit available transport mechanisms to microscale diffusion effectively prevent the delivery of growth factors and survival proteins necessary for cell viability to all but the edges of the biomaterial.

Second, mesoscale architecture is highly influential in determining mechanical properties on the macroscale. In native tissues, strong collagen fibers are oriented within a water-swollen matrix. Native tissues are essentially composite materials containing a cell-enclosing base hydrogel material composed of glycosaminoglycans and reinforcing fibers. Cells are contained within the hydrogel-like material where diffusion is rapid and efficient because of the high water content. Analogous to rebar in concrete, the collagen fibers in the majority of bodily tissues provide most of the tensile mechanical strength of tissues. In addition, the pore sizes and size scale of void spaces within natural materials exist in the mesoscale, ranging from about 0.1 μm for fibrin to about 10 μm for collagen. Thus, the mechanical properties inherent within tissues of the body are generally determined at the mesoscale.

Third, mesoscale architecture provides regions for macromolecular assembly of extracellular matrix components. Controlling mesoscale structure within tissue engineering scaffolds may be used to effectively create differential regions throughout the material that possess dissimilar mechanical, physical, and chemical properties. From a tissue-regeneration perspective, it is valuable to recapitulate this physiologically representative structural diversity, particularly with respect to extracellular matrix components. In materials that have microscale diffusion limitations, there is insufficient free space for the assembly of mesoscale collagenous structures. Even collagen molecules, the smallest subunit of collagen fibers, may be unable to sufficiently diffuse into surrounding material that is subject to these limitations.

Finally, mesoscale internal pore architecture enables the spreading, proliferation, and migration of cells within encapsulant material while simultaneously retaining mechanical properties of the material as a whole.

3. Materials

There are a variety of different materials that may be used to provide the primary and secondary encapsulants in different embodiments. In considering the effect of these different encapsulants, it is useful to compare how the use of an embedded-encapsulation structure compares with the use of a single encapsulation material such as poly(ethylene glycol) (“PEG”), which is an example of a broader class of materials referred to as “hydrogels.” Hydrogels are useful materials in tissue-engineering and tissue-regeneration applications because they provide an environment that is similar to native tissue environments. In tissue-engineering applications, the high water content, facile diffusive properties, resistance to protein adsorption, and tissue-like elasticity that minimizes mechanical or frictional irritation of the surrounding tissue are useful properties of hydrogels. From a cell-delivery standpoint, in situ forming hydrogels lead to excellent homogeneous cell distribution during gel formation.

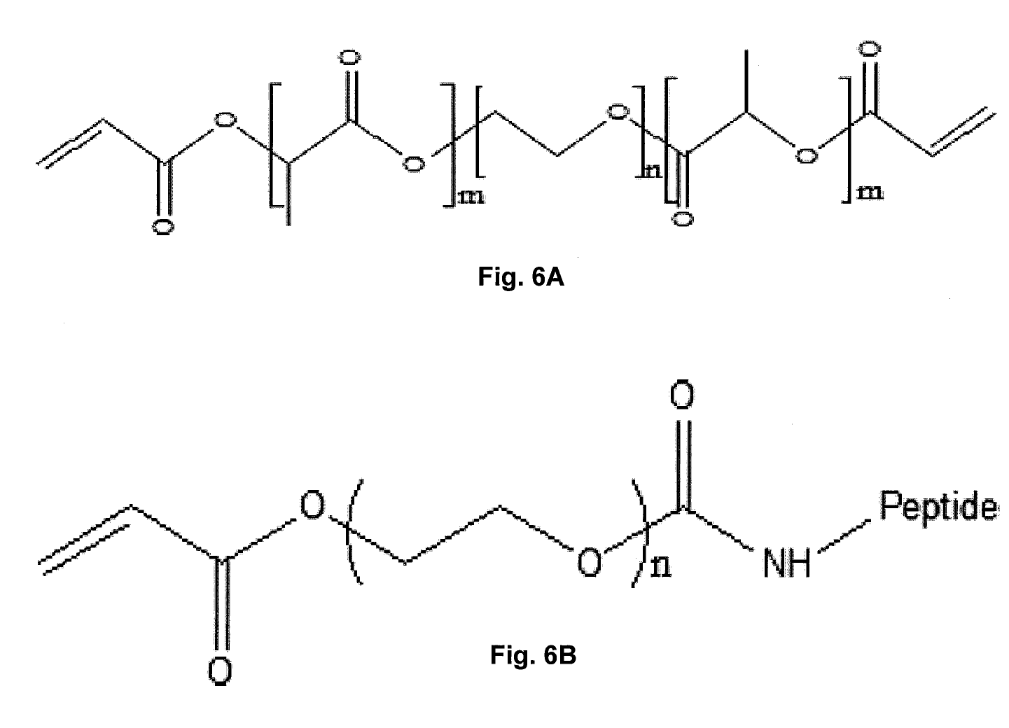

But despite these advantages, there are a number of disadvantages to using monophase hydrogel carriers. The structure of photoreactive PEG is shown in FIG. 6A, and in some instances peptide sequences, growth factors, or hormones may be covalently incorporated within its structure as shown in FIG. 6B. In the presence of a photoinitiating molecule, a free-radical propagation reaction leads to the covalent cross-linking of PEG chains, resulting in a hydrogel. Cell encapsulation results when the reaction is carried out in the presence of cells, and these cells remain viable in these materials over time.

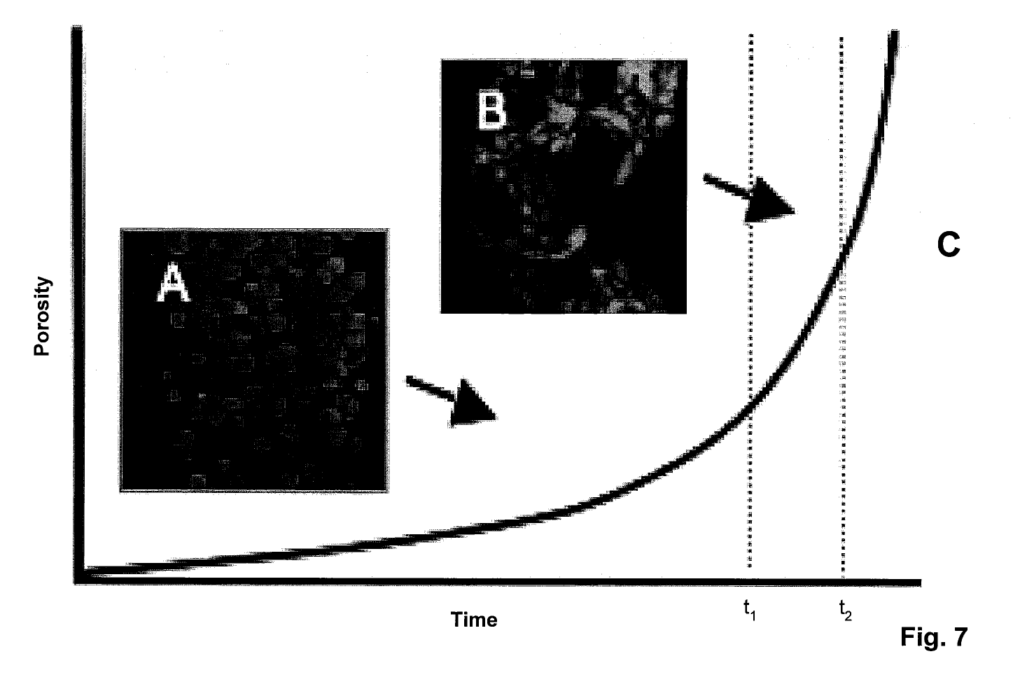

The difficulty of implementing a mesoscale architecture with monophase hydrogels may be better understood with reference to FIG. 7. This drawing shows a degradation profile for a hydrogel by plotting the porosity of the material over time. Because degradable photopolymerizable PEG hydrogels have a network cross-linking density that depends strongly on the extent of degradation, mass loss and porosity of the bulk material increase approximately exponentially with time, with the rate of degradation being dependent on the number of lactic acid repeat units (m in FIG. 6A). Once encapsulated within the PEG hydrogel, cell spreading is initially frustrated due to the small pore sizes. Consequently, the cells retain a substantially spherical morphology, as shown in region A of FIG. 7, up to about time t₁.

As degradation occurs, the porosity gradually increases until the average pore size is large enough that the cell can extend processes and spread out within the gel environment, as depicted in region B of FIG. 7. It is believed that the cells can proliferate in this state, while they are prevented from doing so in the constrained state represented by region A. Unfortunately, while the cells are able to initiate spreading within the gel as it degrades and porosity increases, the bulk material properties are also affected by the increase in mass loss over time. This leads to a dramatic and abrupt disintegration of the entire gel construct as nearly complete digestion is reached in region C of FIG. 7, at about time t₂. The optimal gel state for cell proliferation is maintained for less than 24 hours before biomaterial erosion crosses a critical threshold, loses its mechanical integrity, and releases cells from the matrix into the surrounding environment. The time period between t₁, and t₂ is relatively small, making it difficult to achieve mesoscale architecture with monophase encapsulant.

The implementation of a two-phase or other multiphase material with mesoscale architecture as described above provides regions that allow cells to carry out cellular processes like migration, spreading, proliferation, differentiation, extracellular matrix synthesis and remodeling, and the like. In addition, slowly degrading regions may provide structure and uniform mechanical strength and elasticity over time. In such processes, the primary encapsulant may be selected from a large set of biocompatible, in situ forming hydrogel materials. It is preferable that the material allow cell encapsulation under physiological conditions, i.e. at a temperature near body temperature, at a physiological pH, in an aqueous environment, etc. The material also preferably maintains high cell viability upon encapsulation.

The following lists a variety of materials that may be used as encapsulants. Generally, these materials may be used as either the primary or secondary encapsulant, although as described above, it is generally preferable in constructing the mesoscopic architecture for the primary encapsulant to have a faster degradation profile than the secondary encapsulant.

One material that is particularly suitable for the primary encapsulant includes fibrin glue, which is formed through the reaction of thrombin with fibrinogen. The rate of gelation as well as the structure and mechanical properties of the formed fibrin glue may be altered by changing the relative concentrations of fibrinogen and thrombin, as well as by variations in calcium concentration.

Fibrin glue is of particular interest because of its role in certain blood clots. For instance, upon bone fracture in vivo, a blood clot that has fibrin as its main structural macromolecular component forms. Over time, proliferating osteoprogenitor cells migrate to the area, differentiate to osteoblasts, and repair the bone fracture by replacing the provisional fibrin with a collagenous matrix that is eventually converted to fully functional bone. As a factor involved with initial wound healing events, fibrin glue is thus suitable as a three-dimensional matrix as a component in a composite biomaterial. Fibrinogen is converted to a monomeric form of fibrin through the activation by thrombin, which results in a fibrin clot having adhesive properties. Fibrin glue can thus promote cell adhesion, proliferation, migration, growth, and differentiation as a provisional matrix for tissue regeneration, has been approved by the Food and Drug Administration as a tissue sealant, and acts positively on angiogenesis. Fibrin glue, like other natural extracellular matrices, has advantages over synthetic matrices since natural matrices are capable of locally sequestering, binding, and releasing important growth factors, bioactive molecules, and cell-adhesion proteins through specific protein-matrix interactions. This enables these molecules to be presented to cells in a very natural manner, recapitulating the natural development and cellular processes that occur in vivo.

Another material that is well suited for use as the primary encapsulant is collagen, which may be produced by combining collagen solution with Dulbecco's Modified Eagle's Medium (“DMEM”) and NaOH prior to droplet formation. Collagen solution is available commercially under the brand name PureCol™ from Inamed Corporation. In one exemplary embodiment, the combination with DMEM may be performed at 4° C., with collagen droplets then being heated to about 37° C. for about an hour for the solution to gel.

An alginate/gelatin solution is also well suited for use as the primary encapsulant. Alginate dialdehye (“ADA”) may be synthesized by reacting alginate with sodium metoperiodate in dH₂O overnight in the dark. A solution of ADA containing 0.1 M sodium tetraborate decahydrate may then be combined with a gelatin solution. The two solutions may be emulsified into droplets, which gel on timescales ranging from 20 second to several minutes depending on the relative ADA and gelatin concentrations. Gel structure and cross-linking density may similarly be varied by altering the ADA and/or gelatin concentrations.

In other embodiments, a chitosan solution is prepared with glycerophosphate. Merely by way of example, one appropriate combination uses a 1.5 wt. % solution of chitosan and 135 mM β-glycerophosphate. Separately, hydroxyethyl cellulose (“HEC”) is dissolved in DMEM, with the HEC/DMEM solution then being mixed with the chitosan solution. One suitable combination uses six times the volume of the chitosan solution at 4° C. This solution gels when heated to 37° C. so that droplets may be created and then heated to a temperature of at least 37° C. at the device exit to ensure gelation.

Hyaluronic acid gels may also be used as encapsulants in some embodiments. Hyaluronic acid (HA) may be reacted with methacrylic anhydride (“MA) to create methacrylated hyaluronic acid (“HA-MA”). In the presence of a photoinitiating molecule such as Darocur 2959, an aqueous solution of HA-MA undergoes free-radical cross-linking reactions, resulting in an insoluble gel. Merely by way of example, a suitable concentration of the Darocur 2959 is 0.5 wt. %. Droplets of this material may thus be formed and subsequently polymerized using ultraviolet light to lock in the substantially spherical structure. Material properties may be altered by changing the degree of methacrylation.

Similarly, haparin gels may be synthesized by methacrylating heparin through a reaction with methacrylic anhydride. Cross-linking of this material may similarly be achieved using a photoinitiator and ultraviolet light. Material properties can also be altered by changing the degree of methacrylation.

Another similar material is chondroitin sulfate, which may be methacrylated using methacrylic anhydride to form methacrylated chondroitin sulfate (“CS-MA”). Again, cross-linking of an aqueous CS-MA solution occurs in the presence of a photoinitiator and ultraviolet light, and material properties may be altered by changing the degree of methacrylation.

Dextran is another material that may be methacrylated using methacrylic anhydride. An aqueous solution of dextran-MA may also be photopolymerized using ultraviolet light and a photoinitiator, and material properties may be altered by changing the degree of methacrylation.

While monophase PEG has the concerns discussed above in fabricating a mesoscale architecture, it is well suited for use as the secondary encapsulant. There are, moreover, a number of specific different forms in which it may be provided. For example, hydrolytically degradable lactide ester bonds may be added to the PEG chain terminal OH groups, with the resulting macromer being end-capped with photoreactive methacrylate groups. Under photopolymerization conditions using ultraviolet light an a photoinitiating molecule, an aqueous PEG-LAC-DMA solution is covalently cross-linked. As ester bonds hydrolyze, the network degrades over time.

Alternatively, so-called “Michael-type” PEG may be used. A four-arm tetrafunctionally acrylated PEG molecule can react with a dithiolated PEG molecule to create a cross-linked network in aqueous solution through a Michael-type conjugate addition reaction.

A further example of a suitable encapsulant is poly(propyene fumarate-co-ethylene glycol) (“P(PF-co-EG)”), which is a hydrophilic polymer. At room temperature (25° C.), an aqueous solution of P(PF-co-EG) is liquid, but gels due to phase separation when heated to body temperature (37° C.). As such, a solution of P(PF-co-EG) may be prepared at room temperature, with droplets being formed at room temperature and then heated to body temperature to polymerize. Factors such as the ratio of propylene fumarate to ethylene glycol, molecular weight, and weight fraction in solution all affect material properties and gelation kinetics.

4. Clinical Relevance

As previously noted, there are a variety of clinical settings in which embodiments of the invention find application. For example, the most common type of malignant bone tumor in children and adolescents is osteosarcoma. In many cases, treatment of bone tumors involves the use of chemotherapy followed by resection of the tumor. Tumor resection leaves a bone defect that must be reconstructed. Options for reconstruction include autologous bone grafts, allografts, and/or metallic endoprosthetics. Small defects can be repaired fairly well using nonvascularized autografts from the pelvis or other sites, whereas vascularized autografts such as those taken from the fibula are attractive because the graft usually incorporates better into the defect and may even remodel secondary to the forces exerted across them. Allografts, on the other hand, have no donor site morbidity but have a greater difficulty integrating with the host tissue and require immune suppression therapies. While metallic endoprosthetics provide an immediate method to reconstruct the area, they have a tendency to loosen or fail over time and have a significant risk of infection.

Such treatments may be improved using a cellular matrix like that described above. In particular, in one embodiment, a post-operative bone tumor resection may be followed by injecting homogenously distributed human mesenchymal stem cells in a polyphase encapsulant. The proliferation and spreading of the cells as they develop into osteoblasts with degradation of the encapsulant provides an effective treatment option.

In another example, periodontal disease often results in irreversible bone loss in the alveolar ridge as bacteria trapped beneath the gums secrete acidic byproducts that demineralize the bone. Conventional treatments include the use of antibacterial agents, deep dental cleaning, and various periodontal therapies. Unfortunately, studies on periodontal wound healing have revealed that neither allograft nor autograft procedures result in a true new attachment. A more recently developed approach, guided tissue regeneration (“GTR”) uses various bone graft substitutes, such as a collagen matrix doped with growth-stimulating peptide fragments or growth factors. During GTR, after the periodontal defect is cleaned, the periodontist will often drill into the underlying bone to stimulate blood flow to the area, which brings a source of cells to the defect area. The defect is covered with a GTR membrane, which serves as a barrier between fast-growing soft tissue and the underlying bone defect. The membrane enables slower-growing fibers and bone cells to migrate into the protected area, leading to long-term regeneration of alveolar bone. Studies have shown that GTR-base root coverage can be employed successfully for gingival defects. GTR is among the first successful treatments for regenerating and filling bony defects not based on bone grafts.

Despite the moderate success rates of GTR, in which an average of 50% of the defect is filled in with new bone in 6-12 months, significant hurdles remain with this form of treatment. First, the bone graft substitute is usually derived from bovine bone; consequently, disease transmission concerns have been raised about these materials. Second, the GTR material is a paste that is packed into the socket, which is a relatively invasive form of surgery. Perhaps most significantly, this technique relies on the passive penetration of underlying osteoprogenitor cells into the bone graft substitute material. The limited success of GTR is likely due to the incomplete migration of cells from the material's peripheries as well as the inhomogeneous distribution of cells initially within the implant.

Such treatments may accordingly also be improved using a cellular matrix like that described above. In particular, in one embodiment, a minimally invasive surgery in which a bone graft substitute material used in combination with homogeneously distributed human mesenchymal stem cells is injected and formed in situ. This provides an effective treatment as the stem cells proliferate, spread, and develop into osteoblasts while the encapsulant degrades.

Having described several embodiments, it will be recognized by those of skill in the art that various modifications, alternative constructions, and equivalents may be used without departing from the spirit of the invention. Accordingly, the above description should not be taken as limiting the scope of the invention, which is defined in the following claims.

What is claimed is:

1. A method of producing a cellular matrix for tissue self-assembly, the method comprising:
providing a plurality of encapsulated living cells, wherein each of the living cells is separately encapsulated within a primary encapsulant;
encapsulating the plurality of encapsulated living cells within a secondary encapsulant, wherein the secondary encapsulant is different from the primary encapsulant and comprises a liquid or gel; and
polymerizing the secondary encapsulant.

2. The method recited in claim 1 wherein the living cells are selected from the group consisting of mesenchymal stem cells, chondrocytes, osteoblasts, pancreatic islet cells, neuroprogenitor cells, and mynfibroblasts.

3. The method recited in claim 1 wherein providing the plurality of encapsulated living cells comprises separately microfluidically encapsulating the living cells within the primary encapsulant.

4. The method recited in claim 3 wherein separately microfluidically encapsulating the living cells within the primary encapsulant comprises:
encapsulating each of the living cells within a liquid droplet; and
inducing a phase change of the liquid droplet to produce a solid particle.

5. The method recited in claim 1 wherein the plurality of encapsulated living cells are substantially monodisperse.

6. The method recited in claim 5 wherein the plurality of encapsulated living cells comprise a set of volumes having at least 90% of a size distribution lying within 5% of a median size of the set of volumes.

7. The method recited in claim 1 wherein the plurality of encapsulated living cells comprise a set of substantially spherical volumes, each of the substantially spherical volumes having a diameter between about 10 μm and about 200 μm.

8. The method recited in claim 1 wherein the polymerized second encapsulant comprises an elastic material.

9. The method recited in claim 1 wherein the polymerized secondary encapsulant comprises a solid material.

10. The method recited in claim 1 wherein the primary encapsulant comprises a hydrogel.

11. The method recited in claim 1 wherein the primary encapsulant comprises fibrin glue.

12. The method recited in claim 1 wherein the secondary encapsulant comprises a hydrogel.

13. The method recited in claim 1 wherein the secondary encapsulant comprises poly(ethylene glycol).

14. The method recited in claim 1 further comprising deploying the secondary encapsulant into a living body.

15. The method recited in claim 1 wherein polymerizing the secondary encapsulant comprises photopolymerizing the secondary encapsulant.

16. The method recited in claim 1 wherein polymerizing the secondary encapsulant is selected from the group consisting of thermally polymerizing the secondary encapsulant and chemically polymerizing the secondary encapsulant.

17. The method recited in claim 1 wherein the plurality of encapsulated living cells form an ordered periodic structure within the secondary encapsulant.

18. The method recited in claim 1 wherein the plurality of encapsulated living cells form a two-dimensional periodic structure within the secondary encapsulant.

19. The method recited in claim 1 wherein the plurality of encapsulated living cells form a three-dimensional periodic structure within the secondary encapsulant.

20. The method recited in claim 1 wherein the plurality of encapsulated living cells form a nonperiodic structure within the secondary encapsulant.

21. The method recited in claim 1 wherein:
the plurality of encapsulated living cells comprise a first plurality of a first kind of encapsulated living cells and a second plurality of a second kind of encapsulated living cells;
the second kind of encapsulated living cells is different from the first kind of encapsulated living cells.

22. A method of producing a cellular matrix for tissue self-assembly, the method comprising:
separately microfluidically encapsulating a plurality of living cells within a primary encapsulant, wherein:
the primary encapsulant comprises fibrin glue; and
the plurality of encapsulated living cells comprise a set of substantially spherical volumes having at least 90% of a size distribution lying within 5% of a median size of the set of volumes, each of the substantially spherical volumes having a diameter between about 10 μm and 200 μm;
encapsulating the plurality of encapsulated living cells within a secondary encapsulant, wherein the secondary encapsulant comprises poly(ethylene glycol); and
polymerizing the secondary encapsulant.

23. The method recited in claim 22 wherein the living cells are selected from the group consisting of mesenchymal stem cells, chondrocytes, osteoblasts, pancreatic islet cells, neoroprogenitor cells, and mynfibroblasts.

24. The method recited in claim 22 wherein separately microfluidically encapsulating the living cells within the primary encapsulant comprises:
encapsulating each of the living cells within a liquid droplet; and
inducing a phase change of the liquid droplet to produce a solid particle.

25. The method recited in claim 22 wherein the polymerized secondary encapsulant comprises an elastic material.

26. The method recited in claim 22 wherein the polymerized second encapsulant comprises a solid material.

27. The method recited in claim 22 further comprising deploying the second encapsulant into a living body.

28. The method recited in claim 22 wherein polymerizing the secondary encapsulant comprises photopolymerizing the secondary encapsulant.

29. The method recited in claim 22 wherein polymerizing the secondary encapsulant is selected from the group consisting of thermally polymerizing the secondary encapsulant and chemically polymerizing encapsulating the secondary encapsulant.

30. The method recited in claim 22 wherein the plurality of encapsulated living cells form an ordered periodic structure within the secondary encapsulant.

31. The method recited in claim 22 wherein the plurality of encapsulated living cells form a nonperiodic structure within the secondary encapsulant.

32. A cellular matrix comprising:
a plurality of encapsulated living cells, wherein each of the living cells is separately encapsulated within a primary encapsulant; and
a polymerized secondary encapsulant within which the plurality of encapsulated living cells are disposed, wherein the secondary encapsulant is different from the primary encapsulant.

33. The cellular matrix recited in claim 32 wherein the living cells are selected from the group consisting of mesenchymal stem cells, chondrocytes, osteoblasts, pancreatic islet cells, neuroprogenitor cells, and mynfibroblasts.

34. The cellular matrix recited in claim 32 wherein the plurality of encapsulated living cells are substantially monodisperse.

35. The cellular matrix recited in claim 34 wherein the plurality of encapsulated living cells comprise a set of volumes having at least 90% of a size distribution lying within 5% of a median size of the set of volumes.

36. The cellular matrix recited in claim 32 wherein the plurality of encapsulated living cells comprise a set of substantially spherical volumes, each of the substantially spherical volumes having a diameter between about 10 μm and 200 μm.

37. The cellular matrix recited in claim 32 wherein the polymerized secondary encapsulant comprises an elastic material.

38. The cellular matrix recited in claim 32 wherein the polymerized secondary encapsulant comprises a solid material.

39. The cellular matrix recited in claim 32 wherein the primary encapsulant comprises a hydrogel.

40. The cellular matrix recited in claim 32 wherein the primary encapsulant comprises fibrin glue.

41. The cellular matrix recited in claim 32 wherein the secondary encapsulant comprises a hydrogel.

42. The cellular matrix recited in claim 32 wherein the secondary encapsulant comprises poly(ethylene glycol).

43. The cellular matrix recited in claim 32 wherein the plurality of encapsulated living cells form an ordered periodic structure within the secondary encapsulant.

44. The cellular matrix recited in claim 32 wherein the plurality of encapsulated living cells form a two-dimensional periodic structure within the secondary encapsulant.

45. The cellular matrix recited in claim 32 wherein the plurality of encapsulated living cells form a three-dimensional periodic structure within the secondary encapsulant.

46. The cellular matrix recited in claim 32 wherein the plurality of encapsulated living cells form a nonperiodic structure within the secondary encapsulant.

47. A cellular matrix comprising:
a plurality of encapsulated living cells, wherein:
each of the plurality of living cells is separately encapsulated within a primary encapsulant;
the primary encapsulant comprises fibrin glue; and
the plurality of encapsulated living cells comprise a set of substantially spherical volumes having at least 90% of a size distribution lying within 5% of a median size of the set of volumes, each of the substantially spherical volumes having a diameter between about 10 μm and 200 μm; and
a polymerized secondary encapsulant within which the plurality of encapsulated living cells are disposed, wherein the secondary encapsulant comprises poly(ethylene glycol).

48. The cellular matrix recited in claim 47 wherein the living cells are selected from the group consisting of mesenchymal stem cells, chondrocytes, osteoblasts, pancreatic islet cells, neuroprogenitor cells, and mynfibroblasts.

49. The cellular matrix recited in claim 47 wherein the polymerized secondary encapsulant comprises an elastic material.

50. The cellular matrix recited in claim 47 wherein the polymerized secondary encapsulant comprises a solid material.

51. The cellular matrix recited in claim 47 wherein the plurality of encapsulated living cells form an ordered periodic structure within the secondary encapsulant.

52. The cellular matrix recited in claim 47 wherein the plurality of encapsulated living cells form a nonperiodic structure within the secondary encapsulant.
